(19) United States
(12) Patent Application Publication
(10) Pub. No.: US 2010/0217336 A1
(43) Pub. Date: Aug. 26, 2010

(54) Computerized Planning Tool For Spine Surgery and Method and Device for Creating a Customized Guide for Implantations

(75) Inventors: Neil R. Crawford, Tempe, AZ (US); Nicholas Theodore, Phoenix, AZ (US); Seungwon Baek, Phoenix, AZ (US); Anna G.U. Sawa, Tempe, AZ (US)

Correspondence Address: Husch Blackwell Sanders, LLP;Husch Blackwell Sanders LLP Welsh & Katz, 120 S RIVERSIDE PLAZA, 22ND FLOOR, CHICAGO, IL, 60606, US

(73) Assignee: Catholic Healthcare West (CHW), Phoenix, AZ (US)

(21) Appl. No.: 12/393,605

(22) Filed: Feb. 26, 2009

(51) Int. Cl. A61B 17/56; G06F 19/00; G06F 17/50; G06G 7/48

(52) U.S. Cl. 606/86 R; 700/97; 703/6

(57) ABSTRACT

A system for planning a spine surgery, comprising a haptic interface capable of providing force feedback to the user and a computer adapted to simulate a surgical procedure by responding to inputs from the haptic interface and outputting haptic feedback to the haptic interface is provided.

The system further comprising a rapid prototyping unit including a unit that is adapted to create models of the anatomical region where the surgical procedure will be performed in its current unoperated condition and in the predicted postoperative condition. Further the rapid prototyping unit is adapted to create a three dimensional guide to be used in the surgical procedure as well as suggest revisions to the surgical procedure. The system further comprises a computer that simulates loading of the spine and planned implanted hardware using finite element software.

### CROSS-REFERENCE TO RELATED APPLICATIONS

This invention claims priority to U.S. Provisional Patent Application No. 60/841,469, titled “A Computerized Planning Tool for Spine Surgery”, filed Aug. 31, 2006 and U.S. Provisional Patent Application No. 60/828,039 filed Oct. 3, 2006, titled “Customized Artificial Disc Alignment Guide”. The contents of these applications are incorporated by reference into this application as if fully set forth herein.

### FIELD OF THE INVENTION

This invention generally relates to computerized tool for planning spinal surgery, and, more particularly, for a computerized tool with which a surgeon can perform a simulated surgery using a haptic interface to get an accurate prediction of how to proceed during the actual surgery, suggesting revisions, and creating a three-dimensional model based on the planned procedure. Further, the invention includes the accumulation of data such that the surgeon can, upon completion of the simulated surgery, order a customized guide with which to better perform the surgery by assisting in the implantation. More particularly, the present invention relates to such tools and methods related to spinal surgery, specifically computer aided planning of the surgery and the creation of custom alignment guides for use in implanting discs.

### BACKGROUND OF THE INVENTION

In planning a surgery to stabilize the spine, a surgeon typically examines the patient's diagnostic images, such as x-rays, computed tomography (CT), and magnetic resonance images (MRI), in order to create a surgical plan. The surgical plan that is created based on these images is typically only a rough outline of the actual surgical procedure, as the surgeon is generally unable to accurately predict the reaction of the patient's anatomy in response to the introduction of surgical tools into the body.

For example, a surgeon may decide that a surgical procedure calls for a two-level pedicle screw-rod fixation to be performed, but he will not know prior to surgery exactly where each screw will go, how much distraction will be applied, or what length of screw or rod will be needed. These decisions must be made at the time of surgery and are based on the surgeon's sense of “surgical carpentry”, a skill honed over many years.

In some cases of extreme degeneration or severe injury, the anatomy of the spine may be grotesquely distorted. In these cases, it becomes especially difficult for the surgeon to predict from the preoperative images what he or she can expect to encounter once resections are made and bony structures are realigned. Consequently, the surgeon may use even less of a preoperative plan than in general cases. For example, the surgeon may decide before surgery that the spine will be realigned and fused but leave the decision to what size of graft or how many levels will be included in the fusion to be made during the actual procedure. Giving the surgeon the ability to better predict preoperatively what is to be encountered during surgery would decrease the length of the surgery and lessen the urgency of decision making during the surgical procedure, increasing the surgeon's confidence and the patient's safety.

Several methods have been discussed that attempt to overcome the problems with spine surgery, but none of these methods is an adequate solution.

One visually assistive method that has been implemented in complex spine cases involves the fabrication, via rapid prototyping, of three-dimensional models of the spine from preoperative CT images. Simply holding and manipulating a physical model before and during surgery helps surgeons visualize the anatomy more clearly, quickly, and easily than looking at multiple layers of two-dimensional images on a computer monitor.

However, the usage of three-dimensional models alone has been found to be an inadequate planning tool. Because the models are rigid and have homogenous material properties, the surgeon can practice only limited aspects of surgery with them, such as placing screws or pre-bending plates. An articulated realistic physical model, which responds to drilling and cutting similarly to the actual patient's spine, although more desirable than a rigid model, is likely infeasible since such a model would entail an exorbitant amount of preoperative engineering and fabrication work for each patient.

Another assistive method that has been used is that of creating computer images to illustrate a surgical procedure. In recent years, the ability to semi-automatically convert CT or MRI into images readable by computer-aided design (CAD) and finite element modeling (FEM) software has become commercially available and can be used for the purposes described. However, although a three-dimensional image of the spine can be manipulated with CAD software; such manipulation does not simulate the surgery realistically. Using a mouse to put surgical instrument into place on a CAD model does not accurately reflect the real surgical procedure and therefore does not provide the surgeon with a realistic experience. For example, placing all the screws on an anterior plate with the click of the mouse would ignore the subtle alterations in the plate position that occur as each screw is individually tightened. Further, potentialities for surgical errors may be missed as a result of not having tactile feedback; if a surgeon accidentally tries to drill a screw hole in a trajectory that inadvertently crosses the pedicle wall and violates the spinal canal, such a mistake may go unnoticed if a mouse is used to place the screw, but in an actual procedure this mistake would be evidenced by the sensation of suddenly easier penetration of the drill.

Further, once the surgery is commenced, a common problem with artificial intervertebral discs in the cervical, thoracic, or lumbar spine is placement of the device off-center or improperly angled in the disc space. Such improper alignment can theoretically lead to incorrect loading and kinematics of that motion segment, possibly causing pathological response such as pain, facet fusion, bony bridging of the device, or facet hypertrophy. Usually the surgeon does not realize the alignment is incorrect until a postoperative radiographic image is obtained.

The ProDisc-C® cervical artificial disc, which is one type of artificial disc manufactured by Synthes Spine LLC of Paoli Pennsylvania, requires channels to be rendered thereon for securing the device. The ProDisc-C® system has been engineered so that the surgeon uses tools and methods intended to locate the midline and the appropriate position of the device. However, even after following the correct procedure outlined by the manufacturer, alignment may be off because of asymmetry in the anatomy, difficulty in aligning and interpreting radiographic images, incomplete resection of surrounding soft tissues obscuring full view, or change in path after initial insertion but before final seating of the device. Other spinal implants have similar problems in alignment.

### SUMMARY OF THE INVENTION

We have discovered that a solution to the problem of improper placement of artificial discs and other surgical implants is that of a surface-matched template, unique to each patient, which will enable correct positioning of artificial discs and other surgical implants with minimal effort from the surgeon. These templates are created by existing 3D printers using computers having software of the present invention. The use of haptic devices in interacting with computerized simulations of various surgical devices provides a more realistic simulation of surgery. The system of the present invention system can be customized to the patient on whom the actual surgical procedure will take place, in order to maximize the accuracy of the prediction, as well as provide the surgeon with a guide, for use with the implant, made specifically for the patient thereby better assuring correct alignment of the implant.

Therefore, the present invention contemplates a computerized tool for planning surgery comprising a haptic interface capable of providing force feedback to the user and a computer adapted to simulate a surgical procedure by responding to inputs from the haptic interface and outputting feedback to the haptic interface; and further provides a surgeon with a custom made alignment device created for the particular patient.

The present invention also contemplates a computerized tool for planning surgery comprising a haptic interface capable of providing force feedback to the user; a computer adapted to simulate a surgical procedure by responding to inputs from the haptic interface and outputting feedback to the haptic interface; and a rapid prototyping unit that is adapted to create models of the anatomical region where the surgical procedure will be performed, and/or a rapid prototyping unit adapted to create an alignment device to be used during the actual surgical procedure.

The present invention contemplates a computerized tool for planning surgery comprising a haptic interface capable of providing force feedback to the user and a computer adapted to simulate a surgical procedure by responding to inputs from the haptic interface and outputting feedback to the haptic interface, wherein the computer suggests improvements to the surgeon's plan.

### BRIEF DESCRIPTION OF THE DRAWINGS

The benefits and advantages of the present invention will become more readily apparent to those of ordinary skill in the relevant art after reviewing the following detailed description and accompanying drawings, wherein:

FIG. 1 is a system diagram of the computerized planning tool for spine surgery according to an embodiment of the present invention;

FIG. 2 is a flow chart diagram for general operation of the computerized planning tool for spine surgery according to an embodiment of the present invention.

FIG. 3 is a perspective view of an implantable device of the type requiring alignment in the patient.

FIG. 4 is an x-ray image of the device of FIG. 3 implanted in a patient.

FIGS. 5-14 are schematic representations illustrative of the steps of implantation of an artificial disc using the method of the present invention.

### DETAILED DESCRIPTION OF THE INVENTION

While the present invention is susceptible of embodiment in various forms, there is shown in the drawings and will hereinafter be described a presently preferred embodiment with the understanding that the present disclosure is to be considered an exemplification of the invention and is not intended to limit the invention to the specific embodiment illustrated. It should be further understood that the title of this section of this specification, namely, “Detailed Description of the Invention”, relates to a requirement of the United States Patent Office, and does not imply, nor should be inferred to limit the subject matter disclosed herein.

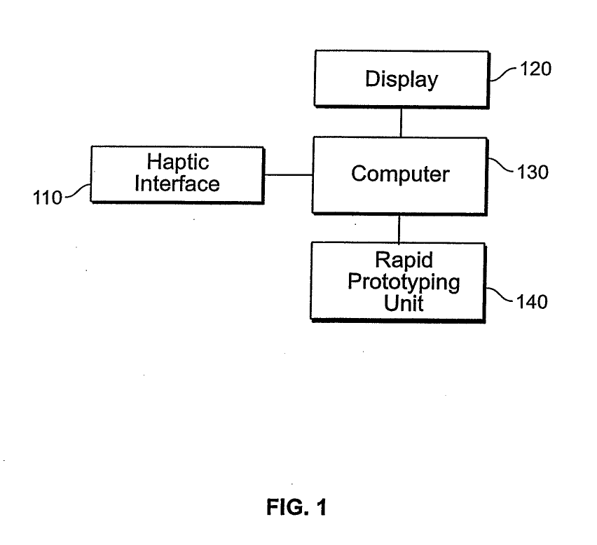

Referring now to FIG. 1, a system diagram of the computerized planning tool for spine surgery according to one embodiment of the invention is shown. FIG. 1 includes a haptic interface 110 that allows the user to interface with computer 130. The simulated surgery is displayed to the user on display 120. Computer 130 is also in communication with rapid prototyping unit 140, which creates prototyped models of the spine in the preoperative and predicted postoperative conditions to assist the surgeon in visualizing the anatomy prior to the actual surgical procedure. Rapid prototyping unit 140 is also capable of creating objects to be used in surgery, such as pedicle screw templates and artificial discs that mirror the 3-D renderings made.

In one embodiment of the invention, a medical image, such as a computed tomography (CT) scan of a spine, is imported from an external source into computer 130. After the user “segments” the medical image so that each vertebra is treated as a different object, this image is then converted to a CAD object. After segmenting, computer 130 can output files in a format importable by various types of FEM software. These files will be referred to as “FEM meshes”.

In an embodiment of the invention, computer 130 automatically assigns different material properties to different elements of the FEM mesh based on the intensity of the voxels of the CT scan. This assigns properties to the material in the surgical simulation that correspond to the properties that material exhibits on the actual patient. For anatomical structures not accurately detected by the CT scan, the software will make assumptions based on normal spine anatomy and programming that reflects the knowledge and know-how of those having surgical and anatomical skill. This allows the surgeon-user to receive feedback from haptic interface 110 consistent with the resistance the surgeon will find during the actual surgical procedure.

In addition, the software is programmed such that many of the consequences that would normally occur or can often occur or have occurred during surgery are accurately reflected in feedback from the haptic device during the course of the simulated surgical procedure, improving the surgical plan. An example of such consequences is a bone fragment resisting realignment because of soft tissue stretching. Persons having ordinary skill in the art will understand that other consequences, the knowledge and experience of which have accumulated over the course of doing such surgical procedures, can be programmed into the software such that almost all eventualities can be seen and considered during the course of planning the procedure.

In one embodiment of the system of the present invention, the system accurately simulates displacement of anatomical material in response to instruments and other apparatus that would be used during the actual surgical procedure. For example, in corpectomy and discectomy surgical procedures, a gap is left in between vertebrae and the surgeon would typically fill the gap with a bone wedge to act as a scaffold for new bone, which will fuse the vertebrae and restore the desired spacing in between the vertebrae. As is known, this distraction of the spine reestablishes proper sagittal curvature. Therefore, in the present embodiment of the invention, the system simulates this procedure so the surgeon can see which bony region serves as a fulcrum and how the bones will become realigned; this behavior may not be immediately apparent to the surgeon when they are in a collapsed state. This information allows the surgeon to distinguish how different amounts of distraction will alter the sagittal plane balance and arrive at the optimal solution in his opinion. After the desired distraction is achieved, the surgeon can specify that the void is to be filled with a graft. This graft can be isolated to determine its exact dimensions, which can be used for rapid prototyping, discussed below.

The surgeon-user performs the simulated surgery using haptic interface 110, and views the simulated surgery on a display 120. The system enables the surgeon to select the shape of the surgical device that corresponds to surgical devices used in the actual surgical procedure; for example the surgeon may choose either a cutting or drilling tip. The system also enables the surgeon to use a variety of virtual tools, such as metal screws, plates, rods, cables, and onlaid bone grafts, which correspond to the tools available to the surgeon during the actual surgical procedure.

After the surgeon performs the simulated surgery, the next step, as is needed, is for the surgical system to suggest any revisions to the procedure. Computer 130 is adaptable to modify and increase the number of revision strategies based on experience and the needs of the surgeon. Examples of revisions the system can make, include but are not limited to pedicle screw trajectory adjustment, anterior plate size and screw trajectory adjustment, and inclusion of adjacent levels within the fusion construct, other revisions are also possible and within the knowledge of persons having ordinary skill in the art. However, the invention does not necessarily include any or all of these revisions, and it is not limited to these types of revisions.

In pedicle screw trajectory adjustment, after the surgeon places the virtual screw in a desirable location, the system will make adjustments based on the desired pedicle screw placement in order to determine whether alternate placement is desired. The computer 130 analyzes the consequences of placing the pedicle screws in each alternate location, in terms of reduced or increased stress to various regions of bone under natural types of loading, and displays these alternate placements and corresponding consequences to the surgeon if desired.

In anterior plate adjustment, the surgeon selects a particular size plate from a set of plates, each with a defined, fixed set of screw holes through which four or more screws would be placed to attach the plate to the vertebral body. Although screws are constrained to specific entry points, their trajectories can be adjusted. After the user defines the plate and screws to be used, and defines the trajectories of the screws within their holes, the computer system will adjust the angle of each screw as was done with the pedicle screw and compute alteration in stresses when loaded in various loading modalities. The system will also analyze the plate in alternate positions, slightly adjusted in relation to the original position, to determine whether a slight alteration is beneficial to stress distributions. As with pedicle screw trajectory permutations, screw repositioning can be performed manually or by automated process.

In inclusion of adjacent levels within the fusion construct, the user selects an adjacent level of pedicle screws or plate adjustment, as described above, and the system performs an analysis at key locations of screw-bone interfaces to determine the peak Von Mises stresses under loading to induce flexion, extension, lateral bending, and axial rotations. This analysis enables the surgeon to determine how much the inclusion of adjacent levels reduces stresses at the level of interest.

In artificial disc placement, the user selects a particular size of artificial disc from a set of artificial discs and places it at the desired position and depth. The system analyzes the sagittal balance that would be induced by the selected artificial disc to determine whether it is optimized. The system also corrects slight malpositioning of the artificial disc to ensure true midsagittal positioning and appropriate anteroposterior positioning to maintain an axis of rotation consistent with the natural axis of rotation of the index level and adjacent levels.

The system will include parameters to ensure that unrealistic results potentially created by the program are not considered. Further, the software can be modified in order to keep the time required to run the simulation low. For example, the mesh size and/or the number of finite element simulations can be decreased to speed up simulation time. Other methods of speeding up the simulation are contemplated and are within the novel scope of the present invention.

In an embodiment of the invention, after analyzing and revising the surgical plan, the system generates a list of all the hardware needed during surgery and the exact dimensions of each piece of hardware. This tends to speed up operating room setup and reduces the need for full sets of hardware to be prepared for surgery. Such lists are also helpful in operating room management, such that the surgical team can monitor and account for all equipment and devices placed in the patient.

A further embodiment of the invention includes a rapid prototyping machine capable of forming a three dimensional model of an anatomical device for use in the implantation process. Rapid prototyped models can be made for the spine segment in its current condition and/or the spine segment after the intended surgery. Further, rapid prototyped models may be made for any bone grafts that are to be shaped for surgery, and any surface-matched drilling templates that are needed during surgery. Allowing the surgeon to view both a model of the spine prior to surgery and a model of the spine after the intended surgery will assist the surgeon in performing the surgery and determining where drilling should take place. The rapid prototyping machine is capable of printing in color, so different anatomical parts and/or surgical tools can be colored differently if desired. It will be understood by persons having ordinary skill in the art that the rapid prototyped models must then be sterilized before being brought into the surgical room.

In an embodiment of the invention, the rapid prototyping machine can also create surface-matched drilling templates if appropriate and desired. In order to combat the potential problem of ill-fitting templates, the present embodiment of the invention contemplates adjusting the contrast of the CT image where the computer identifies the boundary between bone and soft tissue or using alternate bony services as contact points for the templates.

In an alternative embodiment of the invention, an “expert” surgeon uses the computerized tool to create a surgical plan for a “non-expert” surgeon. In this embodiment, the “non-expert”, or “customer”, surgeon sends medical images, such as CT scans, to the expert surgeon. The expert would perform the simulation and then send the customer the rapid prototyped model or models reflecting the desired post-operative condition, the drill guides, the graft sizing templates, and a list of required instrumentation. This level of detailed information would be far more beneficial than the currently-accepted standard in healthcare where surgeons seeking an expert's advice are provided only with a letter describing the recommended surgical solution. Such a system of communication among surgeons on the preferred surgical solution could potentially have a far-reaching impact on the quality of healthcare in spine surgery.

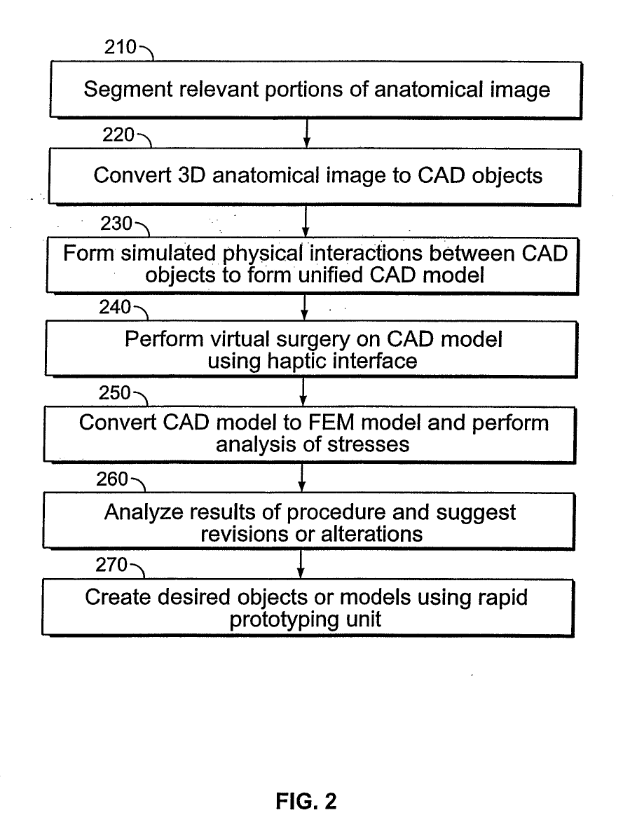

Referring now to FIG. 2, a flow chart showing the general operation of the computerized planning tool for spine surgery according to an embodiment of the present invention is provided. At step 210, a three-dimensional anatomical image, such as a computed tomography (CT) scan, is imported from an external source. This image is segmented so that each vertebra is treated as a different object. At step 220, after segmenting, the relevant sections are converted into computer-assisted design (CAD) images. At step 230, interactions between the individual vertebrae are formed so that the model will react realistically to applied forces. These interactions include the elastic response of ligaments being stretched, intervertebral discs deforming, and of the facet joints colliding. At step 240, the virtual surgical procedure is performed in a manner similar to an actual procedure, except that the procedure is simulated in the computer rather than the patient.

At step 250, the CAD model, now with surgical implants placed, is converted to a FEM model in a format importable by various types of FEM software. At step 260, FEM software analyzes the results of the procedure, and, if desired, revisions are suggested. The surgeon can also make alterations to the procedure to determine their effects on the procedure. For example, the surgeon can alter pedicle screw positioning to see if it would be beneficial. Various means can be used to speed up steps of the procedure, rather than run in real time, such that the surgeon-user can run a number of simulations, using various variables, to more quickly determine the appropriate course of the actual surgery.

Finally, at step 270, any desired objects for use in the surgical procedure can be created, such as templates for screw positioning and the actual operative implants. Also pre-operative and/or post-operative models of the spine may be created as well so that the results of the surgery can be predicted while the patient recovers. Such post-operative examination of a model can aid in determining any follow-up procedures and in determining the appropriate course of post-operative therapy, including physical therapy, to aid the patient's recovery.

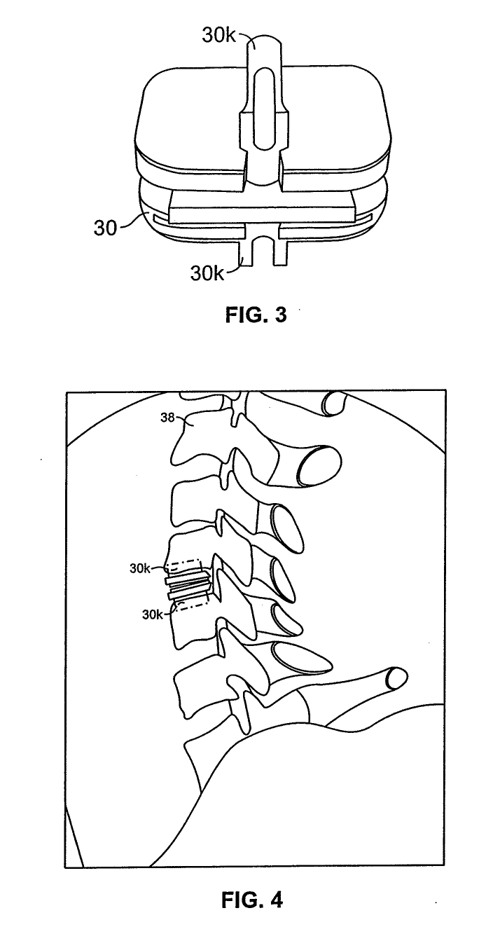

Additionally, as noted above, the invention can include a method of preparing and using a customizable alignment guide for use in the actual surgical procedure. While the steps of such a procedure have been briefly described, the following is a more detailed description of one embodiment. FIGS. 3 and 4 illustrate a typical implant device 30 both prior to and after implantation, FIG. 4 is an X-ray film showing the implant 30 in situ. The implant shown is a ProDisc-C®, as described above, however it will be understood by persons having ordinary skill in the art that other types of implants requiring alignment can be used with the system of the present invention without departing from the novel scope of the present invention.

The present invention provides a means to correctly place and align such a device in the spine and the means to accomplish this is the creation of a disc insertion guide for use by a surgeon during surgery to install the implant. To create a patient-specific disc insertion guide, the following steps would be taken:

A fine-cut (preferably 0.625-mm slice spacing) computed tomography (CT) scan is obtained using standard methods known to persons having skill in the art. Alternately, a magnetic resonance imaging (MRI) scan could be used. As noted above, the CT or MRI scan would be converted to a computer-assisted design (CAD) drawing that is manipulable by standard design software such as Solid Works® made by Solid Works of Concord, Mass. It will be understood by persons having ordinary skill in the art that various software titles are currently commercially available to perform the conversion from CT or MRI to CAD. Examples of conversion software are ScanIP by Simpleware of Exeter, UK or Materialise by Mimics of Leuven, Belgium.

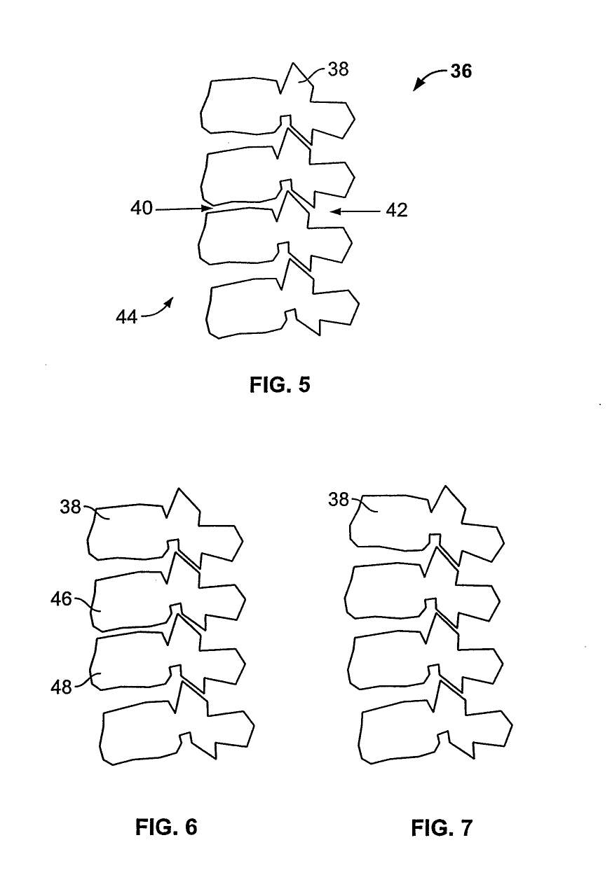

It will be understood that CAD drawing created from CT scans would lack representation of soft tissues (such as native disc) since these tissues are not visible on the CT scan. However, if an MRI scan is used, the soft tissues would be seen and have to be segmented out to provide a clear model of the target body systems. A 3-D CAD model is created of the patient's spine, without intervening soft tissues, in order to best utilize the method of the present invention in creating a usable device. As illustrated in FIG. 5, a sagittal plane representation 36 of the CAD model of the human cervical spine 38 is shown. Note the collapsed disc 40 and improper spinal curvature 42 at the middle segment 44.

The rostral vertebra 46 in FIG. 6, of the motion segment of interest (level to receive the prosthesis 30) and any vertebrae rostral to this one will be segmented so that they are separately manipulable from the caudal vertebra 48 of the motion segment and any vertebrae caudal to this one. A surgeon or technician with a strong understanding of the desired alignment of the artificial disc and surrounding spine would adjust the position and angle of the two segments on the computer monitor so that the optimal spinal alignment is achieved:

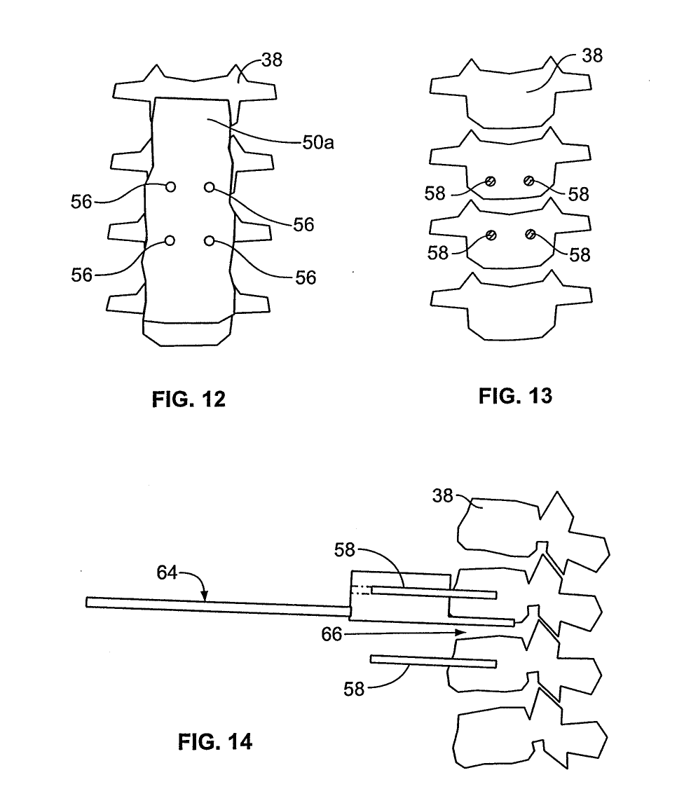

For example, FIG. 6 shows a computer image of the spine prior to adjustment and FIG. 7 shows the same spine after adjustment. Using the computer generated model, as described above, alignment and spacing of the disc would be adjusted from all desired views, including sagittal plane (that shown in the FIG. 6) for correcting kyphosis/lordosis, coronal plane (as shown in FIG. 12) for correcting scoliosis, and possibly transverse plane for correcting axial rotation or lateral subluxation.

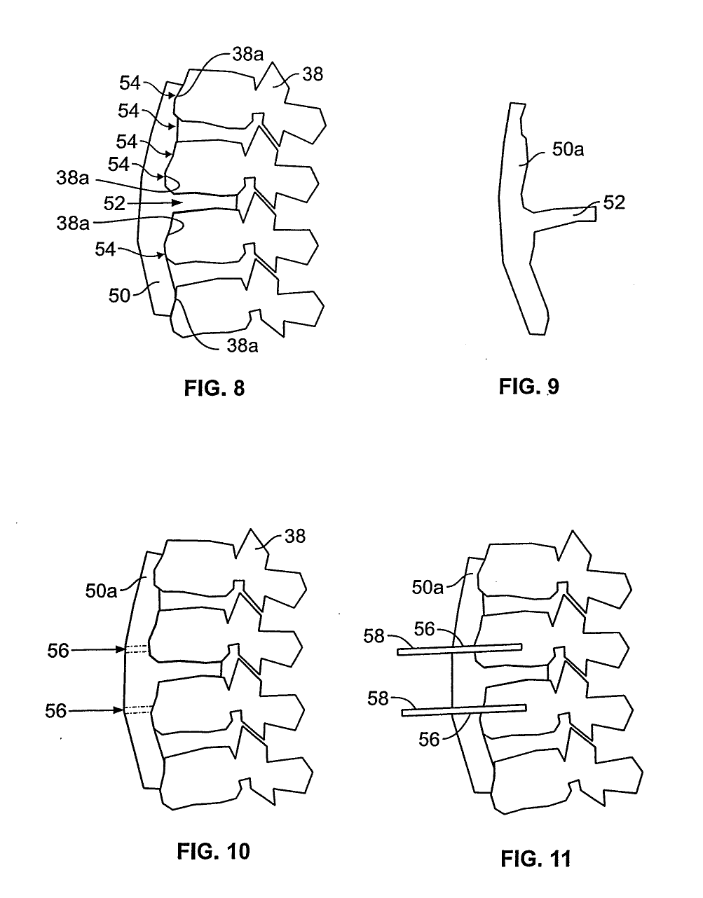

After proper alignment is achieved in the model, a negative (inverse) CAD model 50 (see FIGS. 8 and 9) of the bony anatomy in the disc space and on the anterior surfaces of the vertebrae at (and possibly adjacent to) the level of interest will be created using the CAD software. The negative can include a piece 52 that will act as a full or partial wedge in the disc space and can include extensions 54 that lay on the anterior surface 38a of the spine rostral and caudal to the level of interest. The intention is that this piece 50a, once physically produced, should fit “like a glove” into the disc space and on the front surface of the patient's spine after the native disc has been removed and sufficient soft tissues have been resected from the anterior spinal region.

Holes 56 will be strategically placed in the CAD image of the form fitting plate 50a that will act as guides for the surgeon to place high-precision pilot holes that will later be used to correctly position and insert the artificial disc 30. The location of these holes 56 will depend on the design of the particular artificial disc 30 being used. The holes 56 could be used to accept pins 58 (which could possibly be placed without removing the guide) over which a tool 64 for reaming the channel 66 for a ProDisc-C® keel 30k (see FIG. 3) and flattening the vertebra in the channel adjacent to the disc space would be placed (see FIG. 14). The pins 58 would act as a guide for the position and trajectory of the reaming tool 64 over its path.

After creating the computerized 3D model of the guide plate 50, it could easily be created using a 3D printer such as the ZCorp printer into an actual piece 50a (see FIG. 9). The solid guide 50a, once printed, would need to be made of material that is strong and sterilizable so that it can be used in surgery. If the material to create the guide is difficult to sterilize or work with, an alternative would be to use the 3D printer to create a mold and for plastic, epoxy, or other appropriate solid-forming substance to be poured into the mold to create the guide 50a itself. The guide would be created in advance of the surgery. Then, on the day of surgery, the surgeon would resect the native disc and any other soft tissues that would interfere with proper placement of the customized guide tool. The spine would be distracted and the guide tool would then be inserted in the empty disc space. When distraction is released, the spine should become positioned in the appropriate alignment and the tool should fit snugly in a unique (unambiguous) orientation as shown in FIG. 10. Once in place, the surgeon would use the holes 56 in the guide to drill appropriate pilot holes to be used for artificial disc placement. Pins 58 or other instrumentation may be inserted in the holes if needed. The site preparation (reaming) tool 64 would then be removed and the artificial disc 30 would be inserted.

If the spine is properly prepared, it should be possible for a surgeon to use the guide tool 50a to insert the artificial disc in the correct midline position, without any unwanted angulation, more easily than was previously possible. This tool should save operative time and reduce exposure to x-rays because it eliminates the need to determine the size of the implant and the required amount of distraction intraoperatively. This tool would therefore benefit the quality of healthcare in patients needing disc replacement surgery.

From the foregoing it will be observed that numerous modifications and variations can be effectuated without departing form the true spirit and scope of the novel concepts of the present invention. It is to be understood that no limitation with respect to the specific embodiments illustrated is intended or should be inferred. The disclosure is intended to cover by the appended claims all such modifications as fall within the scope of the claims.

What is claimed is:

1. A system for planning a spine surgery, comprising:
a haptic interface capable of providing force feedback to the user;
a computer adapted to simulate a surgical procedure by responding to inputs from the haptic interface and outputting haptic feedback to the haptic interface.

2. The system of claim 1 further comprising a rapid prototyping unit.

3. The system of claim 2 wherein the rapid prototyping unit is adapted to create models of the anatomical region where the surgical procedure will be performed in the current unoperated condition and the predicted postoperative condition after the planned surgery.

4. The system of claim 2 wherein the rapid prototyping unit is adapted to create a guide to be used in the surgical procedure for accurately positioning surgical implants such as screws or artificial discs.

5. The system of claim 1 wherein the computer is adapted to suggest revisions to the surgical procedure.

6. The system of claim 1 wherein the computer simulates loading of the spine and planned implanted hardware using finite element software.

7. A system for planning spine surgery, comprising:
a haptic interface capable of providing force feedback to the user;
a computer adapted to simulate a surgical procedure by responding to inputs from the haptic interface and outputting haptic feedback to the haptic interface;
a rapid prototyping unit adapted to create a model of the anatomical region where the surgical procedure will be performed in the current preoperative condition and predicted postoperative condition; and
wherein the rapid prototyping unit is adapted to create a guide for use during the surgical procedure.

8. The system for planning spine surgery of claim 7, wherein the guide is created of material capable of being sterilized for surgery.

9. The system for planning spine surgery of claim 8, wherein the guide is created of epoxy material formed from a mold.

10. The system for planning spine surgery of claim 7, wherein the guide can be used as a template for drilling holes into bone for appropriate attachment of the implant in alignment.

11. The system for planning spine surgery of claim 7 wherein the computer is adapted to suggest revisions to the surgical procedure.

12. The system for planning spine surgery of claim 7 wherein the computer simulates loading of the spine and planned implanted hardware using finite element software.

13. A method of surgically placing a spinal implant in alignment, comprising the steps of:
providing a haptic interface capable of providing force feedback to a user;
providing a computer adapted to simulate a surgical procedure by responding to inputs from the haptic interface and outputting haptic feedback to the haptic interface;
providing a rapid prototyping unit, in communication with the computer, adapted to create a three dimensional model of the anatomical region where the surgical procedure will be performed in its current unoperated condition and predicted postoperative condition;
creating a surgical implantation guide using the model created by the rapid prototyping unit; and
using the surgical implantation guide during surgery to guide the configuration and placement of a spinal implant in a patient.

14. The method of surgically placing a spinal implant in alignment of claim 13, including the step of using the guide as a template to drill attachment holes for implant placement as needed.

15. The method of surgically placing a spinal implant in alignment of claim 14, including providing a guide tool and placing guide rods in the attachment holes such that the guide tool can be used to shape bone for the placement of the spinal implant in alignment.
